(19) United States
(12) Patent Application Publication
(10) Pub. No.: US 2022/0357197 A1
(43) Pub. Date: Nov. 10, 2022

(54) LASER SYSTEM MONITORING USING DETECTION OF BACK REFLECTION

(71) Applicant: Alcon Inc., Fribourg (CH)

(72) Inventors: Alireza Malek Tabrizi, Irvine, CA (US); Reza Khazaeinezhad, Lake Forest, CA (US); Adela Apostol, Laguna Hills, CA (US); John Hossein Karim, Cypress, CA (US); Daniel Castro, Lake Forest, CA (US); Corey Stewart, Irvine, CA (US); David Jung, Tustin, CA (US)

(21) Appl. No.: 17/662,135

(22) Filed: May 5, 2022

Related U.S. Application Data
(60) Provisional application No. 63/185,487, filed on May 7, 2021.

(51) Int. Cl. G01J 1/42; G02B 27/14; G01J 1/44; A61F 9/008

(52) U.S. Cl. CPC G01J 1/4257; G02B 27/141; G01J 1/44; A61F 9/0084; G01J 2001/446; A61F 2009/0087; A61F 2009/00887; A61F 2009/00897

(57) ABSTRACT

Systems and methods are disclosed for monitoring a laser system using detection of back reflection. In some embodiments, a laser system comprises a laser, at least one optical fiber, and a back-reflection monitoring sensor for detecting electromagnetic radiation reflected back from the optical fiber(s). The back-reflection monitoring sensor may be adapted to detect back-reflected electromagnetic radiation while the laser system is in use. The laser system may further comprise a computing system adapted to calculate an output power of the system based upon the back-reflected electromagnetic radiation. In some embodiments, a method of monitoring a laser system using detection of back reflection comprises transmitting electromagnetic radiation from a laser, receiving the electromagnetic radiation at one or more optical fibers, and detecting electromagnetic radiation that is back reflected at a back-reflection monitoring sensor.

### TECHNICAL FIELD

The present disclosure is directed to monitoring a laser system, such as a laser system used in ophthalmic procedures.

### BACKGROUND

Lasers are used in many different medical procedures including a number of different ophthalmic procedures. For example, lasers may be used in cataract surgery, such as for fragmenting the cataractous lens. In some procedures, a laser is used for initial fragmentation of the lens, followed by phacoemulsification of the lens by an ultrasonic handpiece to complete the breakdown of the lens for removal. In other procedures, the laser may be used for complete fragmentation or emulsification of the lens for removal, without the need for a separate application of ultrasonic energy. Lasers may also be used for other steps in cataract surgery, such as for making the corneal incision(s) and/or opening the capsule.

Lasers may also be used in vitreoretinal surgery. In some procedures, a laser may be used for vitrectomy, to sever or break the vitreous fibers for removal. The laser may be incorporated into a vitrectomy probe, and the energy from the laser may be applied to the vitreous fibers to sever or break the vitreous fibers for removal.

In other vitreoretinal applications, lasers may be used for photocoagulation of retinal tissue. Laser photocoagulation may be used to treat issues such as retinal tears and/or the effects of diabetic retinopathy.

U.S. Patent Application Publication No. 2018/0360657 discloses examples of an ophthalmic laser system. That application describes laser uses such as for forming surgical cuts or for photodisrupting ophthalmic tissue as well as for cataract surgery, such as laser-assisted cataract surgery (LACS). U.S. Patent Application Publication No. 2019/0201238 discloses other examples of an ophthalmic laser system. That application describes laser uses such as in a vitrectomy probe for severing or breaking vitreous fibers. U.S. Patent Application Publication No. 2018/0360657 and U.S. Patent Application Publication No. 2019/0201238 are expressly incorporated by reference herein in their entirety.

In a laser system, it is desirable to check the components for misalignment and/or defects, such as misalignment of or cracks in optical fibers. In addition, it is desirable to check the output power of a laser system. One current way of checking a laser system is to insert the output tip into a power meter to measure output power and to detect any potential damage. There is a need for improved systems and methods for monitoring a laser system.

### SUMMARY

The present disclosure is directed to improved systems and methods for monitoring a laser system.

In some embodiments, a laser system comprises a laser configured to emit electromagnetic radiation; at least one optical fiber having a proximal end and a distal end, the proximal end of the optical fiber configured to receive electromagnetic radiation from the laser and to transmit electromagnetic radiation from the proximal end to the distal end and out of the distal end of the optical fiber; and a back-reflection monitoring sensor positioned to detect back-reflected electromagnetic radiation reflected back from the at least one optical fiber. The back-reflection monitoring sensor may be a photodiode.

In some embodiments, the laser system may further comprise a beam splitter positioned between the laser and the proximal end of the at least one optical fiber. The beam splitter may be adapted to permit electromagnetic radiation that is transmitted from the laser to pass through the beam splitter to the at least one optical fiber and to direct electromagnetic radiation that is reflected back from the at least one optical fiber to the back-reflection monitoring sensor. Alternatively, the beam splitter may be adapted to direct electromagnetic radiation that is transmitted from the laser to the at least one optical fiber and to permit electromagnetic radiation that is reflected back from the at least one optical fiber to pass through the beam splitter to the back-reflection monitoring sensor.

In some embodiments, at least one optical fiber comprises a delivery optical fiber and an output optical fiber. The output optical fiber may be positioned distal to the delivery optical fiber, and the proximal end of the output optical fiber may be configured to receive electromagnetic radiation from the distal end of the delivery optical fiber.

In some embodiments, the laser system may further comprise a laser housing. The laser may be located inside the laser housing, and at least one optical fiber may be adapted to be removably connected to the laser housing. The back-reflection monitoring sensor may also be located inside the laser housing.

The back-reflection monitoring sensor may be adapted to detect back-reflected electromagnetic radiation while the laser system is in use. The laser system may further comprise a computing system adapted to calculate an output power of the system based upon the back-reflected electromagnetic radiation detected by the back-reflection monitoring sensor.

The laser system may be adapted for performing an ophthalmic procedure. The laser system may be adapted for cataract surgery, for example for fragmenting a cataractous lens. The laser system may be adapted for vitreoretinal surgery, for example for breaking or severing vitreous fibers. The electromagnetic radiation may be in the infrared, visible, or ultraviolet range. In one embodiment, the electromagnetic radiation is in the mid-infrared range.

In some embodiments, a method of monitoring a laser system using detection of back reflection comprises transmitting electromagnetic radiation from a laser in a forward transmission direction to at least one optical fiber; receiving the electromagnetic radiation at the at least one optical fiber, wherein a part of the electromagnetic radiation is transmitted through the at least one optical fiber and another part of the electromagnetic radiation is back reflected from the at least one optical fiber; and detecting electromagnetic radiation that is back reflected from the at least one optical fiber at a back-reflection monitoring sensor. The method may further comprise informing the user of information relating to the detected back reflection. The method may further comprise computing an output power of the laser system from the detected back reflection. The method may further comprise informing the user of the output power of the laser system computed from the detected back reflection. The method may further comprise adjusting an output power of the laser system based upon the detected back reflection.

Further examples and features of embodiments of the invention will be evident from the drawings and detailed description.

### BRIEF DESCRIPTION OF THE DRAWINGS

The accompanying drawings illustrate example implementations of the devices and methods disclosed herein and, together with the description, serve to explain the principles of the present disclosure.

FIG. 1 shows a schematic diagram of an example laser system configured for monitoring using back reflection in accordance with the disclosure.

FIG. 2 shows a flow chart of an example method of monitoring a laser system using detection of back reflection.

The accompanying drawings may be better understood by reference to the following detailed description.

### DETAILED DESCRIPTION

For the purposes of promoting an understanding of the principles of the disclosure, reference will now be made to the implementations illustrated in the drawings, and specific language will be used to describe those implementations and other implementations. It will nevertheless be understood that no limitation of the scope of the claims is intended by the examples shown in the drawings or described herein. Any alterations and further modifications to the illustrated or described systems, devices, instruments, or methods, and any further application of the principles of the present disclosure, are fully contemplated as would normally occur to one skilled in the art to which the disclosure relates. In particular, the features, components, and/or steps described with respect to one implementation of the disclosure may be combined with features, components, and/or steps described with respect to other implementations of the disclosure. For simplicity, in some instances the same reference numbers are used throughout the drawings to refer to the same or like parts.

The terms “proximal” and “distal” are used herein to designate directions or ends of components with reference to the laser source, wherein the proximal direction or end is oriented toward or closer to the laser source and the distal end is oriented away or farther from the laser source. The designations “first” and “second” as used herein are not meant to indicate or imply any particular positioning or other characteristic. Rather, when the designations “first” and “second” are used herein, they are used only to distinguish one component from another. For example, unless otherwise specified, a first optical fiber or a second optical fiber may be positioned closer to the laser source.

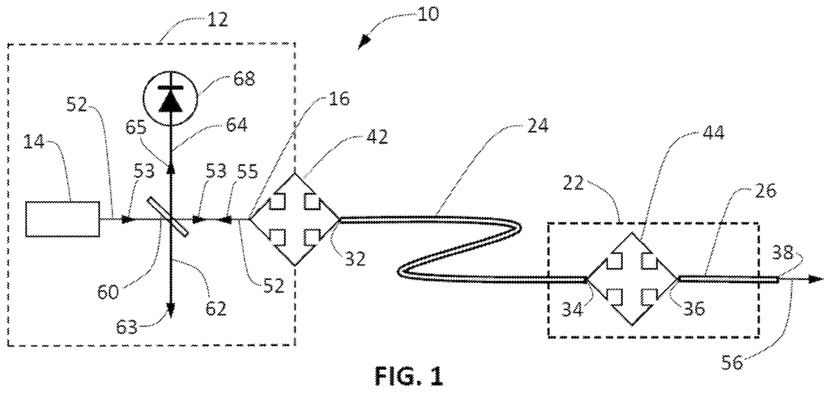

FIG. 1 shows a schematic diagram of an example laser system 10 configured for monitoring using back reflection in accordance with the disclosure. The laser system 10 may be a laser system suitable for one or more ophthalmic procedures. The laser system 10 may be a stand-alone laser system or may be a laser module in an ophthalmic system or console used for ophthalmic procedures.

In some embodiments, the laser system 10 may be suitable for cataract surgery. In some embodiments, the output energy of the laser system is suitable for fragmentation or emulsification a cataractous lens. In some examples, the laser output is used for initial fragmentation of the cataractous lens, followed by phacoemulsification of the lens using an ultrasonic handpiece to complete the breakdown of the lens for removal. In other examples, the laser output is used for fragmentation or phacoemulsification of the lens to a sufficient degree for removal of the lens without the need for a separate application of ultrasonic energy. Additionally or alternatively, the laser output may be suitable for making corneal incisions and/or for opening the lens capsule.

In other embodiments, the laser system may be suitable for vitreoretinal surgery. In some embodiments, the output energy of the laser system is suitable for severing or breaking vitreous fibers for removal. In other vitreoretinal applications, the laser output may be suitable for ophthalmic tissue treatment, such as photocoagulation of retinal tissue to treat issues such as retinal tears and/or the effects of diabetic retinopathy.

As shown in FIG. 1, the laser system 10 includes a laser housing 12, shown schematically as a dashed-line box in FIG. 1. The laser housing 12 houses a laser 14. In addition to the laser 14, other components may be located in the laser housing 12. For example, the laser housing 12 may house components for operating the laser 14, such as a power supply, laser pumps, laser energy control, and monitor. In addition, the laser housing 12 may house components in the optical path of the laser output, such as one or more lenses, mirrors, and optical fibers (not shown).

The laser 14 may be any type of laser suitable for the desired application. The laser 14 may output suitable electromagnetic radiation at any suitable wavelength. For example, the laser 14 may emit electromagnetic radiation in one or more wavelengths in the visible, infrared, and/or ultraviolet wavelengths. The laser 14 may operate or be operated to emit a continuous beam of electromagnetic radiation. Alternatively, the laser 14 may operate or be operated to emit a pulsed beam.

In one example, the laser 14 operates in the infrared range. For example, the laser 14 may output electromagnetic radiation in the mid-infrared range, for example in a range of about 2.0 microns to about 4.0 microns. Some examples wavelengths include about 2.5 microns to 3.5 microns, such as about 2.775 microns, about 2.8 microns, or about 3.0 microns. Such a laser may be suitable, for example, for lens fragmentation in cataract surgery, or for other procedures.

In another example, the laser 14 emits electromagnetic radiation in the ultraviolet range. In another example, the laser 14 emits electromagnetic radiation in the visible range.

The laser system 10 is designed to direct the laser electromagnetic radiation from the laser 14 to an output port 16 of the laser housing 12. In FIG. 1, the output port 16 is indicated schematically as the distal end of the electromagnetic radiation path 52 in the laser housing 12, although it will be understood that an optical component, such as a lens, may be located at the output port 16. The laser system 10 may direct the laser electromagnetic radiation from the laser 14 to the output port 16 through one or more optical components, such as lenses and mirrors.

An instrument 22 may be optically connected to the laser housing 12 to receive the laser electromagnetic radiation from the output port 16. The instrument 22 may be, for example, a handpiece for an ophthalmic procedure. The instrument or handpiece 22 is shown schematically as a dashed-line box in FIG. 1.

The instrument or handpiece 22 may be connected to the laser housing 12 by a delivery optical fiber 24. The delivery optical fiber 24 may be flexible and relatively long to give the operator flexibility in maneuvering the handpiece 22 at some distance away from the laser housing 12. The delivery optical fiber 24 may be, for example, 1 to 3 meters in length. In an example embodiment, the delivery optical fiber 24 may be about 2 meters in length.

The delivery optical fiber 24 may be a part of the handpiece 22, permanently attached thereto. Alternatively, the delivery optical fiber 24 may be removably connected to the handpiece 22. The delivery optical fiber 24 may be permanently or removably connected to the handpiece 22 either directly or through one or more other components.

At its proximal end 32, the delivery optical fiber 24 may be removably connected to the laser housing 12. The delivery optical fiber 24 may have a connector (not shown), such as an SMA connector, that mates with a connector, such as an SMA connector, at the output port 16 of the laser housing 12. Alternatively, the delivery optical fiber 24 may be permanently attached to the laser housing 12. The delivery optical fiber 24 may be permanently or removably connected to the laser housing 12 either directly or through one or more other components.

At the distal end 34 of the delivery optical fiber 24, the delivery optical fiber 24 may be optically coupled to an output optical fiber 26. The output optical fiber 26 has a proximal end 36 and a distal end 38. The distal end output 38 of the output optical fiber 26 constitutes the distal end output of the laser system 10. At its proximal end 36, the output optical fiber 26 may be joined to a connector or ferrule that joins the output optical fiber 26 to the handpiece 22, such that the output optical fiber 26 constitutes a removable part of the handpiece 22. In other embodiments, the output optical fiber may be permanently attached to the rest of the handpiece 22. The output optical fiber 26 may be permanently or removably connected to the rest of the handpiece 22 either directly or through one or more other components. The distal end 34 of the delivery optical fiber 24 may be optically coupled to the proximal end 36 of the output optical fiber 26 either directly or through one or more other components. For example, one or more optical fibers may be positioned between the delivery optical fiber 24 and the output optical fiber 26. One or more other components, such as connectors, lenses, or other components may be positioned between the delivery optical fiber 24 and the output optical fiber 26.

The output optical fiber 26 may be any suitable length. For example, the output optical fiber 26 may be between 20 mm and 100 mm in length. In an example embodiment, the output optical fiber 26 may be about 50 mm in length.

In one example embodiment, the output optical fiber 26 is fixed to a connector or ferrule that can be joined to and removed from the rest of the handpiece 22. The output optical fiber 26 may be a disposable component, such that after use the output optical fiber 26 may be removed from the rest of the handpiece 22 and discarded. A new disposable output optical fiber 26 may be joined to the rest of the handpiece 22 for a subsequent procedure.

The optical fibers in the laser system may be any optical fiber capable of transmitting electromagnetic radiation suitable for the intended application. Any suitable material fiber may be used, including glass fibers or plastic fibers. In one example embodiment, the delivery optical fiber 24 may be a germanium oxide (GeO2) fiber, and the output optical fiber 26 may be a sapphire fiber. Many other examples are possible.

In the embodiment of FIG. 1, a beam splitter 60 is positioned in the laser electromagnetic radiation path 52 between the laser 14 and the output port 16 of the laser housing 12. The beam splitter 60 serves to pass a portion of the laser electromagnetic radiation and to divert a portion of the laser electromagnetic radiation. In the illustrated embodiment, when electromagnetic radiation is emitted from the laser 14 toward the output port 16 along electromagnetic radiation path 52 in the direction of arrows 53, most of the electromagnetic radiation passes through the beam splitter 60 and continues through the output port 16 to the delivery optical fiber 24 and the handpiece 22. A fraction, for example 0.1% to 10%, of the electromagnetic radiation from the laser 14 is diverted by the beam splitter 60 along electromagnetic radiation path 62 in the direction of arrow 63 as a tap signal.

As described in more detail below, depending on the conditions of the laser system 10, some portion of the laser electromagnetic radiation that is transmitted to the components distal to the beam splitter 60, e.g., to the delivery optical fiber 24 and to the output optical fiber 26, gets reflected back toward the laser 14. This back-reflected electromagnetic radiation travels back along electromagnetic radiation path 52 in the direction of arrow 55 to the beam splitter 60. The beam splitter 60 directs a fraction, for example 0.1% to 10%, of this back-reflected electromagnetic radiation along electromagnetic radiation path 64 in the direction of arrow 65, which is the opposite of the arrow 63 along path 62. The back-reflected electromagnetic radiation directed along path 64 in the direction of arrow 65 is directed to a back-reflection monitoring sensor 68 for measuring the back-reflected electromagnetic radiation.

In order to distinguish between the two principal directions of travel, the terms “forward transmission” and “forward-transmitted” are used to refer to electromagnetic radiation transmitted in a direction from the beam splitter 60 and toward the distal end 38 of the output optical fiber 26, i.e., toward the distal end of the laser system 10. The term “back reflection” and “back-reflected” are used to refer to electromagnetic radiation that is reflected back in the opposite direction, from the distal end 38 of the output optical fiber 26 and toward the beam splitter 60, i.e., away from the distal end of the laser system 10.

The back-reflection sensor 68 may be, for example, a photodiode capable of converting the detected electromagnetic radiation into a signal relating to the received electromagnetic radiation. As one example, the back-reflection sensor 68 may be a lead selenide photodetector. Other types of photodetectors and other types of sensors may be used.

In the example of FIG. 1, the beam splitter 60 and the back-reflection sensor 68 are housed within the laser housing 12. In other embodiments, one or both of these components may be located outside of the laser housing 12.

In the illustrated example, the path from the laser 14 to the beam splitter 60 is aligned with the path from the beam splitter 60 to the optical fiber 22, and the path 64 from the beam splitter 60 to the back-reflection sensor 68 is at an angle, e.g., a right angle, to that path. Thus, the beam splitter 60 is adapted to permit electromagnetic radiation that is transmitted from the laser 14 to pass through the beam splitter 60 to the optical fibers 24, 26, and the beam splitter 60 is adapted to direct electromagnetic radiation that is reflected back from the optical fiber 24, 26 to the back-reflection monitoring sensor 68. In an alternative arrangement, the path from the laser 14 to the beam splitter 60 is at an angle, e.g., a right angle, to the path from the beam splitter 60 to the optical fiber 22, and the path 64 from the beam splitter 60 to the back-reflection sensor 68 is aligned with the path from the beam splitter 60 to the optical fiber 22. In this arrangement, the beam splitter 60 is adapted to direct electromagnetic radiation that is transmitted from the laser 14 to the optical fiber(s), and the beam splitter 60 is adapted to permit electromagnetic radiation that is reflected back from the optical fiber(s) to pass through the beam splitter 60 to the back-reflection monitoring sensor 68.

In operation of the example laser system 10 in FIG. 1, the laser 14 is operated to emit electromagnetic radiation, which is transmitted from the laser 14 along the path 52 in the direction of arrows 53 to the output port 16. From the output port 16, the electromagnetic radiation enters the proximal end 32 of the delivery optical fiber 24, travels through the delivery optical fiber 24, and exits the delivery optical fiber 24 at distal end 34. From the distal end 34 of the delivery optical fiber 24, the electromagnetic radiation enters the proximal end 36 of the output optical fiber 26, travels through the output optical fiber 26, and exits output optical fiber 26 at the distal end 38 toward the target site along electromagnetic radiation path 56. The target site may be, for example, a cataractous lens, vitreous fibers, retinal tissue, other ophthalmic tissue, or other tissue in general.

As can be seen in FIG. 1, as the electromagnetic radiation travels from the laser 14 to the distal end 38 of the output optical fiber 26, it traverses locations where a component of the laser systems interfaces with the electromagnetic radiation path. These interfaces include the proximal ends of optical fibers where the electromagnetic radiation enters the optical fibers and the distal ends of optical fibers where the electromagnetic radiation exits the optical fibers. In the example laser system 10 shown in FIG. 1, the laser system includes a first interface location 42 where the electromagnetic radiation exits the laser housing 14 at output port 16 and enters the proximal end 32 of the delivery optical fiber 24 and a second interface location 44 where the electromagnetic radiation exits the distal end 34 of the delivery optical fiber 24 and enters the proximal end 36 of the output optical fiber 26.

The first interface location 42 and the second interface location 44 are illustrated schematically in FIG. 1 as sets of arrows to indicate potential misalignment of one or more components. For example, at the first interface location 42, the proximal end 32 of the delivery optical fiber 24 may be misaligned with the path 52 of the electromagnetic radiation exiting the laser housing 14 at the output port 16. Similarly, at the second interface location 44, the proximal end 36 of the output optical fiber 26 may be misaligned with the distal end 34 of the delivery optical fiber 24.

Misalignment of an optical fiber may be due to misalignment during manufacturing, e.g., misalignment of connected components and/or fiber eccentricity. In embodiments in which the user connects optical fiber components, such as connecting the proximal end 32 of the delivery optical fiber 24 to the laser housing 14 at the output port 16 and connecting the output optical fiber 26 to the rest of the handpiece 22, misalignment of the optical fiber may arise from a misaligned connection. In addition, external factors such as vibrations, temperature changes, etc., may result in misalignment.

In the event that an optical fiber is misaligned, it may result in less than full transmission of the electromagnetic radiation to the target location, as well as some back reflection of the electromagnetic radiation. For example, at the first interface location 42, if the proximal end 32 of the delivery optical fiber 24 is misaligned with the path 52 of the electromagnetic radiation exiting the laser housing 14 at the output port 16, the proximal end 32 of the delivery optical fiber 24 may reflect back some of the electromagnetic radiation. Similarly, at the second interface location 44, if the proximal end 36 of the output optical fiber 26 is misaligned with the distal end 34 of the delivery optical fiber 24, the proximal end 36 of the output optical fiber 26 may reflect back some of the electromagnetic radiation. An increased amount of misalignment results in a decreased amount of transmission to the target location as well as an increased amount of back reflection.

In addition to misalignment, another potential issue that can reduce transmission of electromagnetic radiation to the target location is a crack or break in one of the optical fibers. If an optical fiber is cracked or broken, the crack or break can reduce the intended transmission of electromagnetic radiation and can result in increased back reflection of the electromagnetic radiation.

The back-reflection sensor 68 monitors the laser system 10 for reduced transmission of electromagnetic radiation due to misalignment or optical fiber defects by detecting the amount of back reflection. Increased back reflection due to misalignment and/or optical fiber cracks or breaks results in an increased signal picked up by the back-reflection sensor 68. The system may comprise a computing system, e.g., a processor, memory, and software, firmware and/or hardware, that receives the signals from the back-reflection detector and monitors them. The computing system may include thresholds for assessing when the amount of back reflection is excessive. Such thresholds may be determined based on data for a particular system. The system may inform the user of information relating to the detected back reflection, such as the amount of back reflection, whether a threshold has been crossed, whether misalignment or defects are suspected or detected, or other information relating to the detected back reflection. By monitoring the optical fibers for misalignment and for cracks or breaks, the back-reflection monitoring serves to check the safety of the system. In accordance with embodiments disclosed herein, such monitoring can be done periodically or continuously while the laser system is in use.

In addition to monitoring for misalignment and defects, the back-reflection monitoring can also serve to calibrate the output power of the system. Increased power from the laser will result in increased output power transmitted from the distal end of the system as well as increased back reflection. A correlation can be determined between the output power transmitted from the distal end of the system and the amount of back reflection detected. For example, in some systems, the amount of back reflection detected may be directly proportional to the amount of output power transmitted from the distal end of the system. The correlation between output power and the amount of back reflection may be determined based on data for a particular system.

The laser system's computing system, e.g., processor, memory, and software, firmware and/or hardware, may be configured to calculate the output power transmitted from the distal end of the system based upon the measured back reflection. This calculation may be based upon the correlation determined from data for a particular system. The computer system stores the correlation between the output power transmitted from the distal end of the system and the amount of back reflection detected. While the system is being used, the system may be configured to periodically or continuously measure the back reflection, compute the output power transmitted from the distal end of the system from the measured back reflection, and inform the user of the computed output power. The user can use that information to adjust the power up or down. Additionally or alternatively, the system may automatically adjust the output power of the laser system based upon the detected back reflection.

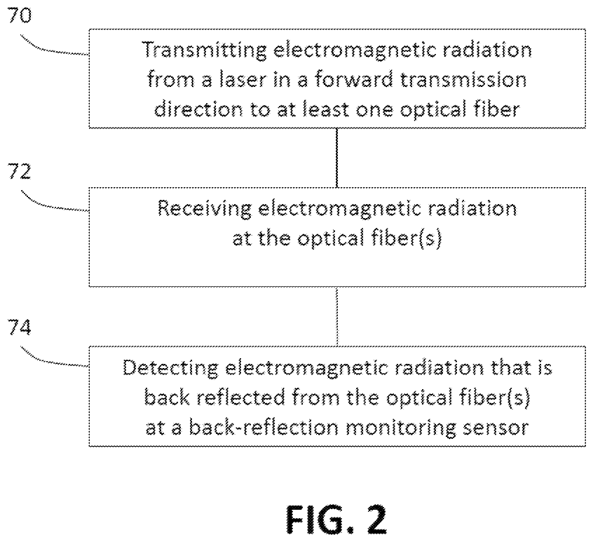

FIG. 2 shows a flow chart of an example method of monitoring a laser system using detection of back reflection. The example method steps shown in FIG. 2 represent only an embodiment, as other variations are possible within the scope of the disclosure.

In step 70, electromagnetic radiation is transmitted from a laser in a forward transmission direction to at least one optical fiber. For example, in the laser system 10 in FIG. 1, electromagnetic radiation is transmitted from the laser 14 in a forward transmission direction to the optical fibers 24, 26.

In step 72, electromagnetic radiation is received at the optical fiber(s). At this step, a part of the electromagnetic radiation is transmitted through the optical fiber(s) and another part of the electromagnetic radiation is back reflected from the optical fiber(s). For example, in the laser system 10 in FIG. 1, electromagnetic radiation is received at the optical fibers 24, 26. Part of the electromagnetic radiation is transmitted through the optical fibers 24, 26, and another part of the electromagnetic radiation is back reflected from the optical fibers 24, 26.

In step 74, electromagnetic radiation that is back reflected from the optical fiber(s) is detected at a back-reflection monitoring sensor. For example, in the laser system 10 in FIG. 1, electromagnetic radiation that is back reflected from the optical fibers 24, 26 is detected at the back-reflection monitoring sensor 68.

After the electromagnetic radiation that is back reflected from the optical fiber(s) is detected at a back-reflection monitoring sensor, the method may include informing the user of information relating to the detected back reflection, for example on a display. The method may include computing an output power of the laser system from the detected back reflection. The method may include informing the user of the output power of the laser system computed from the detected back reflection, for example on a display. The method may include adjusting an output power of the laser system based upon the detected back reflection, automatically by the system, or manually by the user. The adjustments may be made continuously or at intervals.

As would be understood by persons of ordinary skill in the art, systems and methods as disclosed herein have advantages over prior systems and methods. For example, in some prior systems and methods, a laser system is checked by inserting the output tip into a power meter to measure output power and to detect any potential damage. When the laser system is in use, such as in cataract surgery or vitreoretinal surgery, the output tip is in use and cannot be checked using such a power meter. Thus, if there is a drop in output power, for example due to misalignment or damage, it would go undetected during use. In addition, if the output tip were withdrawn to be checked using a power meter, it would create sterility issues with respect to continued use of the output tip. By contrast, systems and methods as described herein, with monitoring using detection of back reflection, can monitor the laser output power and/or can monitor for damage while the laser system is in use. In addition, in some embodiments, systems and methods as described herein can be used for feedback for adjustment of output power, either automatically or manually, during a procedure. The back-reflection monitoring sensor can be used intermittently or continuously to get intermittent or continuous readings during a procedure.

Persons of ordinary skill in the art will appreciate that the embodiments encompassed by the disclosure are not limited to the particular example embodiments described above. While illustrative embodiments have been shown and described, a wide range of modification, change, and substitution is contemplated in the foregoing disclosure. It is understood that such variations may be made to the foregoing without departing from the scope of the disclosure. Accordingly, it is appropriate that the appended claims be construed broadly and in a manner consistent with the disclosure.

What is claimed is:

1. A laser system comprising:
a laser configured to emit electromagnetic radiation;
at least one optical fiber having a proximal end and a distal end, the proximal end of the optical fiber configured to receive electromagnetic radiation from the laser and to transmit electromagnetic radiation from the proximal end to the distal end and out of the distal end of the optical fiber; and
a back-reflection monitoring sensor positioned to detect back-reflected electromagnetic radiation reflected back from the at least one optical fiber.

2. The laser system as recited in claim 1, wherein the back-reflection monitoring sensor is a photodiode.

3. The laser system as recited in claim 1, further comprising a beam splitter positioned between the laser and the proximal end of the at least one optical fiber.

4. The laser system as recited in claim 3, wherein the beam splitter is adapted to permit electromagnetic radiation that is transmitted from the laser to pass through the beam splitter to the at least one optical fiber, and wherein the beam splitter is adapted to direct electromagnetic radiation that is reflected back from the at least one optical fiber to the back-reflection monitoring sensor.

5. The laser system as recited in claim 3, wherein the beam splitter is adapted to direct electromagnetic radiation that is transmitted from the laser to the at least one optical fiber, and wherein the beam splitter is adapted to permit electromagnetic radiation that is reflected back from the at least one optical fiber to pass through the beam splitter to the back-reflection monitoring sensor.

6. The laser system as recited in claim 1, wherein the at least one optical fiber comprises a delivery optical fiber and an output optical fiber each having a proximal end and a distal end, wherein the output optical fiber is positioned distal to the delivery optical fiber, and wherein the proximal end of the output optical fiber is configured to receive electromagnetic radiation from the distal end of the delivery optical fiber.

7. The laser system as recited in claim 1, further comprising a laser housing, wherein the laser is located inside the laser housing, and wherein the at least one optical fiber is adapted to be removably connected to the laser housing.

8. The laser system as recited in claim 7, wherein the back-reflection monitoring sensor is located inside the laser housing.

9. The laser system as recited in claim 1, wherein the back-reflection monitoring sensor is adapted to detect back-reflected electromagnetic radiation while the laser system is in use.

10. The laser system as recited in claim 1, wherein the laser system further comprises a computing system adapted to calculate an output power of the system based upon the back-reflected electromagnetic radiation detected by the back-reflection monitoring sensor.

11. A laser system for performing an ophthalmic procedure, the laser system comprising:
a laser configured to emit electromagnetic radiation;
at least one optical fiber having a proximal end and a distal end, the proximal end of the optical fiber configured to receive electromagnetic radiation from the laser and to transmit electromagnetic radiation from the proximal end to the distal end and out of the distal end of the optical fiber; and
a back-reflection monitoring sensor positioned to detect back-reflected electromagnetic radiation reflected back from the at least one optical fiber;
wherein the back-reflection monitoring sensor is adapted to detect back-reflected electromagnetic radiation while the laser system is in use during the ophthalmic procedure.

12. The laser system as recited in claim 11, wherein the laser system is adapted for fragmenting a cataractous lens.

13. The laser system as recited in claim 11, wherein the electromagnetic radiation is in the mid-infrared range.

14. A method of monitoring a laser system using detection of back reflection, the method comprising:
transmitting electromagnetic radiation from a laser in a forward transmission direction to at least one optical fiber;
receiving the electromagnetic radiation at the at least one optical fiber, wherein a part of the electromagnetic radiation is transmitted through the at least one optical fiber and another part of the electromagnetic radiation is back reflected from the at least one optical fiber; and
detecting electromagnetic radiation that is back reflected from the at least one optical fiber at a back-reflection monitoring sensor.

15. The method of monitoring a laser system using detection of back reflection as recited in claim 14, further comprising:
informing the user of information relating to the detected back reflection.

16. The method of monitoring a laser system using detection of back reflection as recited in claim 14, further comprising:
computing an output power of the laser system from the detected back reflection.

17. The method of monitoring a laser system using detection of back reflection as recited in claim 16, further comprising:
informing the user of the output power of the laser system computed from the detected back reflection.

18. The method of monitoring a laser system using detection of back reflection as recited in claim 14, further comprising:
adjusting an output power of the laser system based upon the detected back reflection.

19. The method of monitoring a laser system using detection of back reflection as recited in claim 14, wherein the at least one optical fiber comprises a delivery optical fiber and an output optical fiber each having a proximal end and a distal end, wherein the output optical fiber is positioned distal to the delivery optical fiber, and wherein the proximal end of the output optical fiber is configured to receive electromagnetic radiation from the distal end of the delivery optical fiber.

20. The method of monitoring a laser system using detection of back reflection as recited in claim 14, wherein the back-reflection monitoring sensor detects back-reflected electromagnetic radiation while the laser system is in use.
